(19) United States
(12) Patent Application Publication
(10) Pub. No.: US 2026/0273241 A1
(43) Pub. Date: Sep. 17, 2026

(54) SCORING BALLOON CATHETER

(71) Applicant: DK MEDICAL TECHNOLOGY CO., LTD., Suzhou, Jiangsu (CN)

(72) Inventors: Yulin WENG, Suzhou (CN); Dongzi NIU, Suzhou (CN); Weiwei LI, Suzhou (CN)

(73) Assignee: DK MEDICAL TECHNOLOGY CO., LTD., Suzhou, Jiangsu (CN)

(21) Appl. No.: 18/852,215

(22) PCT Filed: Jul. 25, 2024
(86) PCT No.: PCT/CN2024/107510

§ 371 Date: May 3, 2026

(30) Foreign Application Priority Data
Apr. 28, 2024 (CN) 202410519258.6

(51) Int. Cl. A61M 25/10; A61M 25/09

(52) U.S. Cl. CPC A61M 25/104; A61M 25/09; A61M 2025/09008; A61M 2025/1086

(57) ABSTRACT

A scoring balloon catheter provided by the present application includes a catheter seat, an inner tube, an outer tube, a balloon body, a scoring member, and a torsion guide wire. The outer tube is sleeved on an outer side of the inner tube. A proximal end of the outer tube is connected to the catheter seat, and a distal end of the outer tube is connected to the balloon body. The scoring member is arranged on an outer wall of the balloon body in an axial direction. Two ends of the torsion guide wire are connected to the balloon body and the catheter seat respectively. The torsion guide wire is located on the inner tube, or located between the inner tube and the outer tube, or located on the outer tube. The scoring member and the torsion guide wire are located on the same plane of radial section. In this case, torque of the catheter seat may be transmitted to the balloon body by arranging the torsion guide wire on the inner tube or the outer tube, or between the inner tube and the outer tube. Besides, since the torsion guide wire and the scoring member are located on the same plane of radial section, accurate alignment of the scoring member with a lesion location may be effectively realized, and a lesion is directionally cut by the scoring member, so that an effect of stenosis dilatation of a vessel during an angioplasty process is greatly improved. In addition, a variety of structural designs are performed on the scoring member to make it have very good flexibility.

### CROSS-REFERENCE TO RELATED APPLICATIONS

The present application claims priority to the Chinese patent application No. 202410519258.6 filed on Apr. 28, 2024 to the China Patent Office, and entitled “SCORING BALLOON CATHETER”, the entire content of which is incorporated herein by reference.

### TECHNICAL FIELD

The present application belongs to the technical field of medical devices, and particularly relates to a scoring balloon catheter.

### BACKGROUND

Over the years, artery occlusion in a human body has become a major medical problem. This is due to the fact that the artery occlusion results in a reduced blood flow passing through an artery, which leads to a variety of serious complications. An artery stenosis is primarily due to deposits of cholesterol, calcium, and fibrotic tissue, and an angioplasty is probably the most commonly used treatment. An angioplasty process involves dilating an occluded artery with an expandable balloon. However, this treatment option suffers from a problem of a high rate of restenosis.

To solve the above problem, a scoring balloon has been developed. A scoring member on the balloon can be used to cut a stenosis during the angioplasty process to enhance the efficacy of stenosis dilatation.

However, most artery stenoses are formed as eccentric lesions, i.e., a lesion does not extend completely around a circumference of an affected body vessel, which results in a fact that the existing scoring balloon cannot efficiently align the scoring member with a lesion location accurately, and is not very effective for stenosis dilatation of the eccentric lesions.

Therefore, how to realize the accurate alignment of the scoring member with the lesion location, so as to make the effect of stenosis dilatation better, is an urgent problem to be solved by those of skill in the art.

### SUMMARY OF THE INVENTION

An objective of the present application is to provide a scoring balloon catheter, which may effectively realize accurate alignment of a scoring member with a lesion location, so as to make an effect of stenosis dilatation better.

In order to solve the above technical problem, the present application provides a scoring balloon catheter, including: a catheter seat, an inner tube, an outer tube, and a balloon body. The outer tube is sleeved on an outer side of the inner tube. A proximal end of the outer tube is connected to the catheter seat, and a distal end of the outer tube is connected to the balloon body.

The scoring balloon catheter further includes: a scoring member and a torsion guide wire. The scoring member is arranged on an outer wall of the balloon body and is oriented in an axial direction parallel to the balloon body. The torsion guide wire is located in an interlayer, on an outer side wall, or on an inner side wall of the inner tube. Or, the torsion guide wire is located between the inner tube and the outer tube. Or, the torsion guide wire is located in an interlayer, on an outer side wall, or on an inner side wall of the outer tube. The scoring member and the torsion guide wire are located on the same plane of radial section.

Optionally, in the above scoring balloon catheter, a plurality of scoring members are provided.

A plurality of torsion guide wires are in one-to-one correspondence with the scoring members. Or, the number of the torsion guide wires is one or more. The one or more torsion guide wires are in one-to-one correspondence with part of the scoring members.

Optionally, in the above scoring balloon catheter, at least one imaging ring is arranged at a distal end of the torsion guide wire.

And/or, at least one imaging ring is arranged on the scoring member.

And/or, at least one imaging ring is arranged on the inner tube at a position corresponding to the scoring member.

Optionally, in the above scoring balloon catheter, the torsion guide wire is a variable-diameter guide wire, and a diameter of the variable-diameter guide wire gradually decreases from a proximal end thereof to a distal end thereof.

Optionally, in the above scoring balloon catheter, the scoring member is of a toothed rack structure with a serrated surface on a side away from the balloon body.

Optionally, in the above scoring balloon catheter, the scoring member has an L-shaped cross-section. And/or, a plurality of slots are formed at intervals in a surface on a side of the scoring member facing the balloon body. The slots are configured to be bonded to the balloon body by a glue.

Optionally, in the above scoring balloon catheter, the scoring member is provided with a break point in a lengthwise direction. A thickness of the scoring member at the break point is less than a thickness at other parts thereof where no break point is arranged.

Optionally, in the above scoring balloon catheter, the scoring member is of a helical spring structure.

Optionally, in the above scoring balloon catheter, when the torsion guide wire is located in the interlayer, on the outer side wall, or on the inner side wall of the inner tube, the torsion guide wire and the inner tube are of an integrally formed structure or bonded together.

When the torsion guide wire is located in the interlayer, on the outer side wall, or on the inner side wall of the outer tube, the torsion guide wire and the outer tube are of an integrally formed structure or bonded together.

When the torsion guide wire is located between the inner tube and the outer tube, two ends of the torsion guide wire are connected to the balloon body and the catheter seat respectively, and part or a whole of the torsion guide wire is connected to the outer side wall of the inner tube.

Optionally, in the above scoring balloon catheter, the balloon body is of a structure with a cylindrical middle part and two tapered end parts, the cylindrical middle part is a working segment, and the two tapered end parts are non-working segments.

The scoring member is arranged on the cylindrical middle part of the balloon body.

When a plurality of scoring members are provided, the plurality of scoring members are evenly distributed in a circumferential direction on an outer surface of the balloon body.

The present application provides the scoring balloon catheter with the beneficial effects that:
torque of the catheter seat may be transmitted to the balloon body by arranging the torsion guide wire on the inner tube or the outer tube, or between the inner tube and the outer tube, besides, since the torsion guide wire and the scoring member are located on the same plane of radial section, accurate alignment of the scoring member with a lesion location may be effectively realized, and a lesion is directionally cut by the scoring member, so that an effect of stenosis dilatation of a vessel during an angioplasty process is greatly improved. In addition, a variety of structural designs are further performed on the scoring member to make it have very good flexibility.

### BRIEF DESCRIPTION OF THE DRAWINGS

In order to explain technical schemes in embodiments of the present application or the prior art more clearly, accompanying drawings that need to be used in descriptions of the embodiments or the prior art will be briefly introduced below. Apparently, the accompanying drawings in the following descriptions are only embodiments of the present application, and for those of ordinary skill in the art, on the premise of no creative work, other accompanying drawings may further be obtained from the provided accompanying drawings.

FIG. 1 is a schematic structural diagram of a scoring balloon catheter provided by an embodiment of the present application.

FIG. 2 is a partially enlarged diagram at A of FIG. 1.

FIG. 3 is a local sectional diagram of FIG. 2.

FIG. 4 is a partially enlarged diagram at B of FIG. 3.

FIG. 5 is a sectional view at C-C of FIG. 3.

FIG. 6 is a schematic structural diagram of a variable-diameter guide wire provided by an embodiment of the present application.

FIG. 7 is a schematic structural diagram of a first scoring member before a bottom is bent provided by an embodiment of the present application.

FIG. 8 is a partially enlarged diagram at D of FIG. 7.

FIG. 9 is a schematic diagram of a local structure of a first scoring member after a bottom is bent provided by an embodiment of the present application.

FIG. 10 is a sectional view of a first scoring member provided by an embodiment of the present application.

FIG. 11 is a schematic structural diagram of a second scoring member provided by an embodiment of the present application.

FIG. 12 is a partially enlarged diagram at E of FIG. 11.

FIG. 13 is a schematic structural diagram of a fourth scoring member provided by an embodiment of the present application.

FIG. 14 is a partially enlarged diagram at F of FIG. 13.

In the above accompanying drawings:
1—balloon body; 2—scoring member; 3—inner tube; 4—outer tube; 5—catheter seat; 501—guide wire guiding opening; 502—balloon pressure filling and depressurizing connector; 6—torsion guide wire; 7—imaging ring;
a—guide wire cavity; and b—balloon filling cavity.

### DETAILED DESCRIPTION

Embodiments of the present application are described in detail below, and examples of the embodiments are shown in accompanying drawings, wherein the same or similar reference numerals throughout indicate the same or similar elements or elements having the same or similar functions. The embodiments described below with reference to the accompanying drawings are exemplary, are only used for explaining the present application, and cannot be construed as a limitation to the present application.

In the description of the present application, it needs to be understood that terms “proximal end” and “distal end” described throughout refer to proximity and distance with respect to an operator. When the present application is used, the end close to a doctor or operator is the “proximal end”, i.e., an end where the operator is located. The end away from the doctor or operator is the “distal end”, i.e., an end where a balloon is located. The above involving orientation descriptions is only for the convenience of describing the present application and simplifying the description, rather than indicating or implying that an apparatus or element referred to must have a specific orientation and be constructed and operated in a specific orientation, thus cannot be understood as a limitation to the present application.

A core of the present application is to provide a scoring balloon catheter, which may effectively realize accurate alignment of a scoring member with a lesion location, so as to make an effect of stenosis dilatation better.

In order to enable those of skill in the art to better understand the technical schemes provided by the present application, the present application will be further illustrated in detail below in conjunction with the accompanying drawings and detailed embodiments.

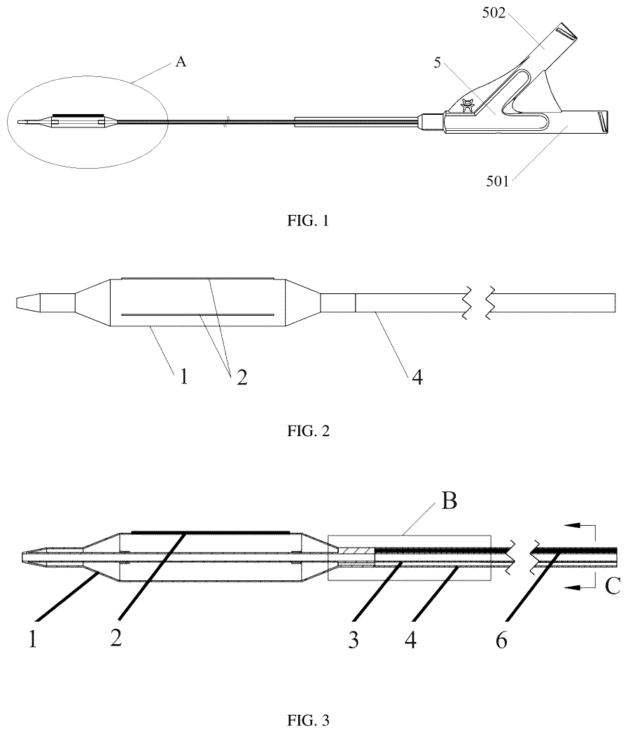

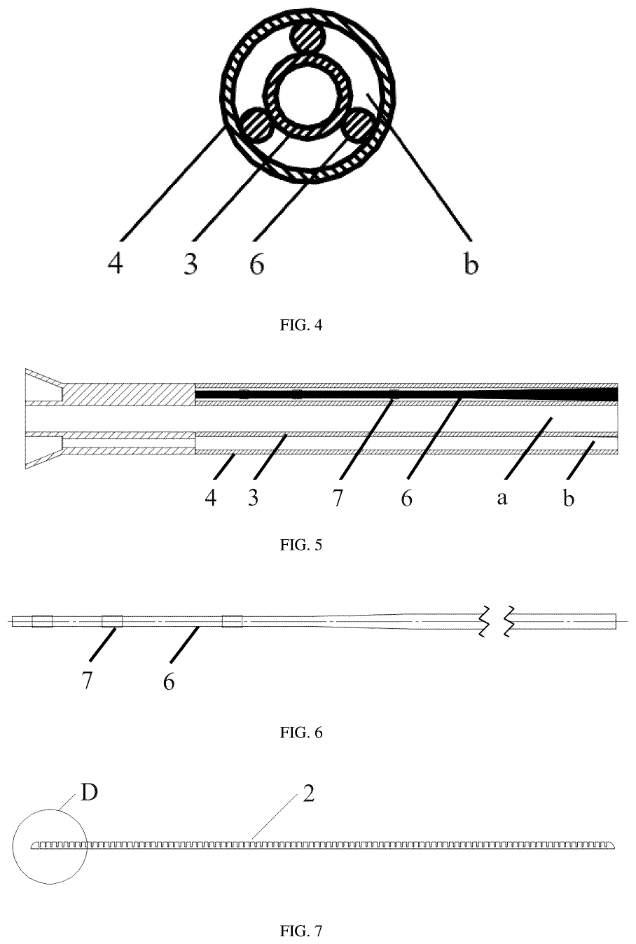

Specifically, referring to FIG. 1-FIG. 14, an embodiment of the present application discloses a scoring balloon catheter. In this embodiment, the scoring balloon catheter includes a catheter seat 5, an inner tube 3, an outer tube 4, a balloon body 1, a scoring member 2, and a torsion guide wire 6. Typically, a distal end of the balloon body 1 may further be provided with a tail end tube according to needs.

The catheter seat 5 is of a hollow structure, and is provided with a balloon pressure filling and depressurizing connector 502 and a guide wire guiding opening 501.

The outer tube 4 is sleeved on an outer side of the inner tube 3. Specifically, the tail end tube, the balloon body 1, and the outer tube 4 are sequentially sleeved on the inner tube 3 from far to near. An inner cavity of the inner tube 3 is a guide wire cavity a. The guide wire cavity a communicates with the guide wire guiding opening 501. In use, a guide wire is guided to enter the guide wire cavity a of the scoring balloon catheter from the tail end tube and is guided out of a body through the guide wire guiding opening 501. Guiding the guide wire can aid in a delivery of an interventional device based on the scoring balloon catheter.

A proximal end of the outer tube 4 is connected to the catheter seat 5. A distal end of the outer tube 4 is provided with the balloon body 1. A balloon filling cavity b is formed between an inner wall of the outer tube 4 and an outer wall of the inner tube 3. The balloon pressure filling and depressurizing connector 502 communicates with an inner cavity of the balloon body 1 through the balloon filling cavity b and is configured to fill and expand or depressurize and contract the balloon body 1.

The scoring member 2, as a member for cutting a lesion location, is specifically arranged on an outer wall of the balloon body 1 and is oriented in an axial direction of the balloon body 1. In view of a problem in the prior art that a scoring balloon cannot effectively align the scoring member 2 with the lesion location accurately, in particular, the torsion guide wire 6 is additionally arranged.

Specifically, the torsion guide wire 6 may be arranged in an interlayer, on an outer side wall, or on an inner side wall of the inner tube 3. The torsion guide wire 6 may also be arranged between the inner tube 3 and the outer tube 4 according to needs. The torsion guide wire 6 may further be arranged in an interlayer, on an outer side wall, or on an inner side wall of the outer tube 4. It needs to be noted that those of skill in the art may select an arrangement location of the torsion guide wire 6 according to actual needs, and this embodiment does not limit the specific arrangement location of the torsion guide wire 6.

The scoring member 2 should be located on the same plane of radial section as the torsion guide wire 6, such that a location where the scoring member 2 is may be determined by observing the location of the torsion guide wire 6. In addition, the torsion guide wire 6 may also increase an anti-torsion capacity of the scoring balloon catheter, so that torque may be transmitted from the torsion guide wire 6 to the balloon body 1 by twisting the catheter seat 5, and thus the scoring member 2 is directionally twisted and accurately aligned with the lesion location to be cut.

It needs to be noted that when the torsion guide wire 6 is arranged between the inner wall of the outer tube 4 and the outer wall of the inner tube 3, the torsion guide wire 6 may transmit 100% of torque of the proximal end to the balloon body 1, the torsion guide wire 6 extends in a space between the outer tube 4 and the inner tube 3, one end of the torsion guide wire 6 is located on a side where the catheter seat 5 is located, and the other end of the torsion guide wire 6 may extend to a proximal end of the balloon body 1, so that by twisting the catheter seat 5, the balloon body 1 may be made to rotate along under the action of the torque transmitted by the torsion guide wire 6. The two ends of the torsion guide wire 6 need to be fixed to the inner tube 3 and the outer tube 4 respectively, and part or a whole of the torsion guide wire 6 needs to be connected to the outer side wall of the inner tube 3 (i.e., a middle part of the torsion guide wire 6 also needs to be fixed, e.g., the whole of the torsion guide wire 6 is fixed to the outer side wall of the inner tube, or, a part or a plurality of spaced parts on the torsion guide wire 6 are fixed to the outer side wall of the inner tube).

When the torsion guide wire 6 is arranged on the inner tube 3, specifically, it may be connected to an inner wall or the outer wall of the inner tube 3 (the two may be of an integrally formed structure or be connected in manners such as bonding). The torsion guide wire 6 may also be arranged in an intermediate layer of the inner tube 3 (i.e., the torsion guide wire 6 is arranged between the outer wall and the inner wall of the inner tube 3, so as to enable the inner tube 3 to have a role of transmitting the torque).

When the torsion guide wire 6 is arranged on the outer tube 4, specifically, it may be connected to the inner wall or an outer wall of the outer tube 4 (the two may be of an integrally formed structure or be connected in manners such as bonding). Or the torsion guide wire 6 is located in an intermediate layer of the outer tube 4 (i.e., the torsion guide wire 6 is arranged between the outer wall and the inner wall of the outer tube 4, so as to enable the outer tube 4 to have a role of transmitting the torque).

In summary, according to the scoring balloon catheter provided by the present application, the torque of the catheter seat 5 may be transmitted to the balloon body 1 by arranging the torsion guide wire 6 on the inner tube 3 or the outer tube 4, or between the inner tube 3 and the outer tube 5. Besides, since the torsion guide wire 6 and the scoring member 2 are located on the same plane of radial section, the location of the scoring member 2 may be judged by observing the location of the torsion guide wire 6 at the proximal end, accurate alignment of the scoring member 2 with the lesion location is effectively realized, and a lesion is directionally cut by the scoring member 2, so that an effect of stenosis dilatation of a vessel during an angioplasty process is greatly improved.

When the number of the scoring member 2 is one, correspondingly, the number of the torsion guide wire 6 is also one. When a plurality of scoring members 2 are provided, the plurality of scoring members 2 are distributed in a circumferential direction of the balloon body 1. In one form, a plurality of torsion guide wires 6 are in one-to-one correspondence with the scoring members 2, i.e. the torsion guide wires 6 and the scoring members 2 are the same in number and are in corresponding locations. In another form, a plurality of torsion guide wires 6 are provided, and the plurality of torsion guide wires 6 are in one-to-one correspondence with part of the scoring members 2. That is, the number of the torsion guide wires 6 is smaller than the number of the scoring members 2, then the torsion guide wires 6 may realize a location correspondence with part of the scoring members 2 according to a preset condition, and the preset condition may be a location relationship or a mode conducive to an operation, etc. For example, if four scoring members 2 are provided, in the circumferential direction of the balloon body 1, one scoring member 2 is oriented at an interval of 90°. In this embodiment, only two torsion guide wires 6 may be oriented, these two torsion guide wires 6 correspond to two of the scoring members 2 respectively, e.g. one of the torsion guide wires 6 may correspond to one of the scoring members 2, and the other torsion guide wire 6, after being spaced apart by 180°, corresponds to the other scoring member 2. An operator may directly predict without doubt that a location between the two torsion guide wires 6 further has a corresponding scoring member 2, i.e. the location of the scoring member 2 may also be judged by a reasonable prediction, even without the torsion guide wires 6.

The torsion guide wires 6 and the scoring members 2 on the balloon body 1 are located on a straight line and are in one-to-one correspondence, and the torsion guide wires 6 may be one or more, depending on the number of the scoring members 2.

In a specific embodiment, the torsion guide wire 6 may be made of a metal material with a better torque transmitting capacity, and the outer tube 4 may be made of a transparent material. The outer tube 4 is transparent, so that when the torsion guide wire 6 is arranged on the outer tube 4, or is arranged between the inner tube 3 and the outer tube 4, naked eyes of the operator may see the torsion guide wire 6 at the proximal end. If the torsion guide wire 6 is arranged on the inner wall or in the interlayer of the inner tube 3, the torsion guide wire 6 may further be observed by designing the inner tube 3 to be made of a transparent material. Of course, the torsion guide wire 6 can also be observed by marking the location of the torsion guide wire 6 at the proximal end of the outer tube 4, without the need to design the outer tube 4 to be made of a transparent material.

Further, in order to determine the location of the scoring member 2 more intuitively, a distal end of the torsion guide wire 6 is provided with at least one imaging ring 7, and the imaging ring 7 is configured to display the location of the torsion guide wire 6 in a body of a patient. Since the torsion guide wire 6 and the scoring member 2 are located on the same straight line, the operator determines the location of the scoring member 2 by observing the location of the torsion guide wire 6. It needs to be noted that at least one imaging ring 7 is arranged on the scoring member 2, and/or, at least one imaging ring 7 is arranged on the inner tube 3 at a position corresponding to the scoring member 2. That is, the torsion guide wire 6, the scoring member 2, and a position of the inner tube 3 corresponding to the scoring member 2, these three locations may all be provided with imaging rings 7, or part of them may be selected to be provided with the imaging rings 7, and this embodiment does not limit the specific locations of the imaging rings 7, as long as the location of the scoring member 2 can be indicated.

When the scoring balloon catheter disclosed by the embodiment of the present application is in use, the balloon body 1 in a contracted state is first delivered to the lesion location of a vessel. When the location of the scoring member 2 needs to be adjusted, the catheter seat 5 may be rotated to drive the outer tube 4 and the inner tube 3 to rotate, which in turn drives the balloon body 1 and the scoring member 2 to rotate. The location of the torsion guide wire 6 is observed, and the rotation is made until the torsion guide wire 6 and the lesion location are located on the same plane of radial section. Since the torsion guide wire 6 and the scoring member 2 are located on the same plane of radial section, at this time, the scoring member 2 is aligned with the lesion location, then the balloon body 1 is filled and expanded through the balloon pressure filling and depressurizing connector 502 via the balloon filling cavity b, and the scoring member 2 is in contact with the lesion and cuts it directionally.

On the basis of the above specific implementation, the torsion guide wire 6 may be a variable-diameter guide wire, and a diameter of the variable-diameter guide wire gradually decreases from a proximal end (located on the side of the catheter seat 5) thereof to a distal end (located on the side of the balloon body 1) thereof. An advantage of the torsion guide wire 6, as a torsion member, arranged in the form of a variable diameter, is that a diameter of the proximal end is larger, enabling the torsion guide wire 6 to fulfill a role of transmitting the torque, and a diameter of the distal end is smaller, enabling the distal end to have better flexibility and facilitating the distal end to pass through the tortuous vessel. In addition, a thickness of the variable-diameter guide wire may further be adjusted according to an actual loading situation, thus improving a bearing capacity. At the same time, material consumption may also be reduced, a manufacturing cost may be reduced, and the performance and economy of a mechanical design may be improved.

Specifically, the balloon body 1 is of a structure with a cylindrical middle part and two tapered end parts, the cylindrical middle part is a working segment, i.e., a part capable of playing a role of vasodilation, and the two tapered end parts are non-working segments. An outer surface of the inner tube 3 at a position corresponding to the balloon body 1 is provided with an imaging ring 7. The balloon body 1 may be localized to the lesion location by the imaging ring 7.

The balloon body 1 has a diameter size of 1.0 mm-40.0 mm and a length of 10 mm-300 mm. The specific size of the balloon body 1 may be selected according to an actual application scenario. This embodiment does not limit the diameter and length size of the balloon body 1.

A material of the balloon body 1 includes one or more of polyvinyl chloride, polyethylene, polyurethane, polyamide, polyether block polyamide, polyethylene terephthalate, or a high polymer material with better biocompatibility. The material of the balloon body 1 may be the same as a material commonly used in the prior art, the focus of this embodiment does not lie herein, and those of skill in the art may select a material that meets requirements according to actual needs.

The scoring member 2 may be fixed to a surface of the cylindrical middle part of the balloon body 1. The scoring member 2 is oriented in an axial direction parallel to the balloon body 1, and may typically be fixed by means of glue sticking. The number of the scoring members 2 is n, and n is an integer greater than or equal to 1. When a plurality of scoring members 2 are provided, they may be uniformly distributed in a circumferential direction on an outer surface of the balloon body 1, and any two adjacent scoring members 2 are spaced apart in the circumferential direction by an included angle of 360°/n. Optionally, the number of the scoring members 2 is 3, and any two adjacent corresponding scoring members 2 are spaced apart in the circumferential direction by an included angle of 120°.

The scoring member 2 may be adaptively structurally improved according to actual needs, as follows in a variety of design forms.

In a first form, the scoring member 2 is of a toothed rack structure with a serrated surface on a side away from the balloon body 1. As shown in FIG. 7 and FIG. 8, the surface on the side of the scoring member 2 away from the balloon body 1 is serrated. The serrated structure is a plurality of tooth portions arranged at intervals, with this structure, the flexibility of the scoring member 2 may be improved, and the scoring member 2 more easily passes through the tortuous vessel. A groove is formed between every two adjacent tooth portions. A width of each groove may be 0.05 mm-0.2 mm. A depth of each groove may be 0.05 mm-0.4 mm. An interval between the grooves may be 0.1 mm-2 mm. It may be understood by those of skill in the art that forming of the grooves increases the flexibility of the scoring member 2, so that the grooves may provide a space for the displacement of the tooth portions when the scoring member 2 is deformed. Moreover, since the grooves have a certain depth, a thickness corresponding to a part where the grooves are formed is smaller, and deformation is more prone to occurring here, facilitating the passage of the scoring member 2 through the tortuous vessel. Width and depth sizes of the grooves may be set according to needs.

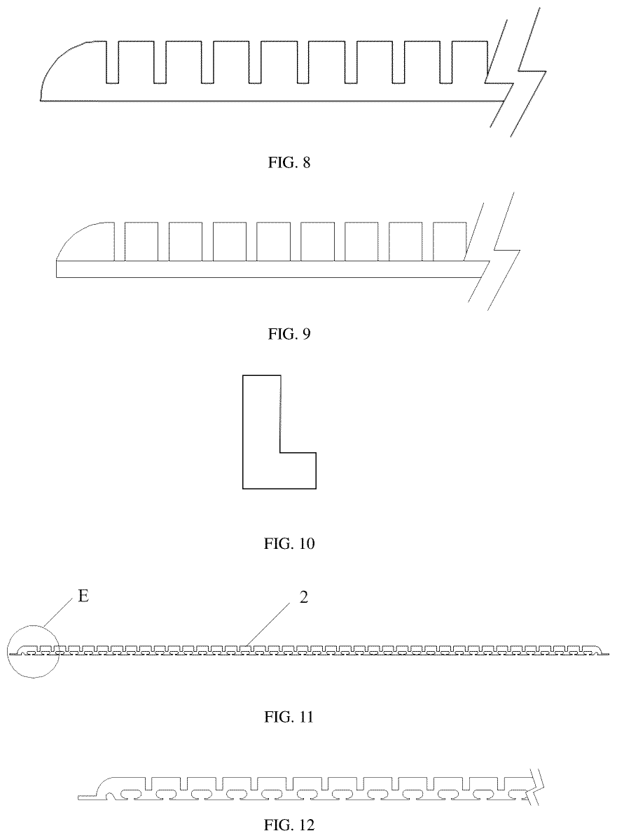

The scoring member 2 has an L-shaped cross-section, i.e., as shown in FIG. 7 and FIG. 8. Bending is performed at about 0.1 mm-0.2 mm from a bottom edge of the scoring member 2 to form an “L” shaped bent structure as shown in FIG. 9 and FIG. 10, and thus the side of the scoring member 2 facing the balloon body 1 has a larger surface area, so that a contact area between the scoring member 2 and a surface of the balloon body 1 may be increased, enabling the scoring member 2 to be more firmly bonded to the surface of the balloon body 1.

In a second form, the surface on the side of the scoring member 2 away from the balloon body 1 is serrated. The serrated structure is a plurality of tooth portions arranged at intervals, with this structure, the flexibility of the scoring member 2 is better, and the scoring member 2 more easily passes through the tortuous vessel. A groove is formed between every two adjacent tooth portions. A depth of each groove may be 0.2 mm-0.5 mm. A width of each groove may be 0.05 mm-0.2 mm. A depth of each groove may be 0.05 mm-0.3 mm. An interval of the grooves may be 0.1 mm-2 mm.

A plurality of slots are formed at intervals in a surface on a side of the scoring member 2 facing the balloon body 1, and the slots are configured to be bonded to the balloon body 1 by a glue. As shown in FIG. 11 and FIG. 12, the slots may be T-shaped holes, and a plurality of T-shaped holes are formed at intervals in a bottom of the scoring member 2. Locations of the T-shaped holes should be arranged to correspond to the tooth portions between the grooves, i.e., the T-shaped holes should avoid the locations of the grooves in a height direction to prevent thicknesses of bottoms of the grooves from being too small, which affects the strength of the scoring member 2. Because the T-shaped holes are formed in the side facing the balloon body 1, the glue may enter the T-shaped holes during bonding by the glue. After the glue dries, a physical snap structure exists between the dried glue and the scoring member 2, which will be firmer than in the first form where bonding is performed by the glue only.

On the basis of the first form, in order to further improve the flexibility of the scoring member 2, a third form may be designed (no accompanying drawing is provided). The scoring member 2 is of a strip-shaped structure with a serrated surface on a side away from the balloon body 1. Bending is performed at a bottom edge of the scoring member 2 to form an “L” shaped bent structure. At the same time, the scoring member 2 is provided with a break point in a lengthwise direction, and a thickness of the scoring member 2 at the break point is less than a thickness at other parts thereof where no break point is arranged.

Similarly, on the basis of the second form, a fourth form may be designed. The scoring member 2 is of a strip-shaped structure with a serrated surface on a side away from the balloon body 1. A plurality of slots are formed at intervals in a surface on a side of the scoring member 2 facing the balloon body 1, and the slots are configured to be bonded to the balloon body 1 by a glue. At the same time, the scoring member 2 is provided with a break point in a lengthwise direction, and a thickness of the scoring member 2 at the break point is less than a thickness at other parts thereof where no break point is arranged.

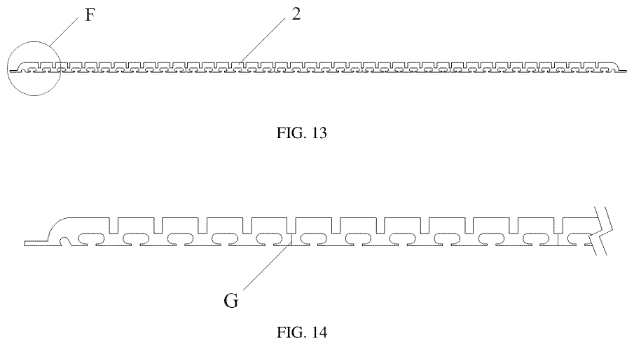

As shown in FIG. 13 and FIG. 14, some break points are added in the middle of the scoring member 2, and a wall thickness at the break points may be one-quarter to three-fourths of a thickness at other locations of the scoring member 2. A plurality of break points may be arranged in the lengthwise direction of the scoring member 2, an interval between two adjacent break points may be designed to be 1 mm-10 mm, and a specific interval size may be selected according to a total length of the scoring member 2. In this embodiment, the design of the break points may improve the flexibility of the scoring member 2, and the break points and the serrated structure may improve a bending capacity of the scoring member 2 in different directions, which improves a capacity of passing through the tortuous vessel.

On the basis of the first, second, third and fourth forms above, further, two ends of the scoring member 2 may adopt a circular arc design to prevent scratching of the vessel. The scoring member 2 is made of a sheet metal material such as stainless steel, and the above grooves and slots may all be formed by laser cutting.

In a fifth form, the scoring member 2 is of a helical spring structure. The scoring member 2 adopts the helical spring structure wound from a flat wire, and a cross section of the scoring member 2 after winding is an isosceles triangle (a size of a vertex angle is less than 60 degrees) (the cross section may further be other polygonal structures). When the scoring member 2 is wound by using a nickel-titanium flat wire, the interval may be 0-0.076 mm, which is not shown in the figure. By using this structure, the flexibility of the scoring member 2 may also be improved, and the material of the scoring member 2 is a nickel-titanium alloy, which has good shape memory performance.

In summary, the scoring balloon catheter disclosed by the embodiment of the present application has the following beneficial effects.
1) The variable-diameter guide wire is arranged between the inner tube 3 and the outer tube 4 and is configured to transmit the torque, so that the role of adjusting the location of the scoring member 2 may be realized by rotating the catheter seat 5.
2) The variable-diameter guide wire and the scoring member 2 are located on the same plane of radial section, so that the location of the scoring member 2 may be known by observing the location of the variable-diameter guide wire, and a purpose of directional cutting is realized.
3) A variety of structural designs are performed on the scoring member 2 to make it have very good flexibility so as to adapt to the tortuous vessel.

As shown in the present application and claims, unless the context clearly suggests an exception, words “one”, “a”, “a kind of”, and/or “the”, etc. do not refer specifically to the singular, but may also include the plural. In general, terms “include” and “contain” suggest only the inclusion of clearly identified steps and elements, these steps and elements do not constitute an exclusive list, and a method or device may also contain other steps or elements. An element defined by a phrase “including a . . . ” does not exclude the existence of other identical elements in the process, method, commodity or device including the element.

In the descriptions of the embodiments of the present application, unless otherwise indicated, “/” denotes the meaning of or, e.g., A/B may denote A or B. “And/or” here merely describes the association relationship of associated objects, which means that there may be three kinds of relationships, for example, A and/or B may mean that there are three kinds of situations: A alone, A and B at the same time, and B alone. Further, in the descriptions of the embodiments of the present application, “a plurality of” means two or more than two.

In the descriptions of the present application, unless otherwise expressly limited, words such as arrangement, installation, and connection shall be broadly construed, and those of skill in the art may reasonably determine specific meanings of the above words in the present application in conjunction with specific contents of the technical schemes.

In this specification, each embodiment is described in a progressive mode, and each embodiment focuses on the differences from other embodiments. The same and similar parts among various embodiments may be referred to each other.

Specific examples are applied herein to illustrate the principle and implementations of the present application, and the above illustrations of the embodiments are only used for helping to understand the method of the present application and its core idea. It should be pointed out that for those of ordinary skill in the art, without departing from the principle of the present application, several improvements and modifications may further be made on the present application, and these improvements and modifications also fall within the protection scope of the claims of the present application.

1. A scoring balloon catheter, comprising: a catheter seat (5), an inner tube (3), an outer tube (4), and a balloon body (1), wherein the outer tube (4) is sleeved on an outer side of the inner tube (3), a proximal end of the outer tube (4) is connected to the catheter seat (5), and a distal end of the outer tube (4) is connected to the balloon body (1);
wherein the scoring balloon catheter further comprises: a scoring member (2) and a torsion guide wire (6), wherein the scoring member (2) is arranged on an outer wall of the balloon body (1) and is oriented in an axial direction parallel to the balloon body (1), the torsion guide wire (6) is located in an interlayer, on an outer side wall, or on an inner side wall of the inner tube (3), or, the torsion guide wire (6) is located between the inner tube (3) and the outer tube (4), or, the torsion guide wire (6) is located in an interlayer, on an outer side wall, or on an inner side wall of the outer tube (4), and the scoring member (2) and the torsion guide wire (6) are located on the same plane of radial section.

2. The scoring balloon catheter according to claim 1, wherein a plurality of scoring members (2) are provided; and
a plurality of torsion guide wires (6) are in one-to-one correspondence with the scoring members (2), or, the number of the torsion guide wires (6) is one or more, and the one or more torsion guide wires (6) are in one-to-one correspondence with part of the scoring members (2).

3. The scoring balloon catheter according to claim 1, wherein at least one imaging ring (7) is arranged at a distal end of the torsion guide wire (6);
and/or, at least one imaging ring (7) is arranged on the scoring member (2);
and/or, at least one imaging ring (7) is arranged on the inner tube (3) at a position corresponding to the scoring member (2).

4. The scoring balloon catheter according to claim 1, wherein the torsion guide wire (6) is a variable-diameter guide wire, and a diameter of the variable-diameter guide wire gradually decreases from a proximal end thereof to a distal end thereof.

5. The scoring balloon catheter according to any one of claims 1-4, wherein the scoring member (2) is of a toothed rack structure with a serrated surface on a side away from the balloon body (1).

6. The scoring balloon catheter according to claim 5, wherein the scoring member (2) has an L-shaped cross-section, and/or, a plurality of slots are formed at intervals in a surface on a side of the scoring member (2) facing the balloon body (1), and the slots are configured to be bonded to the balloon body (1) by a glue.

7. The scoring balloon catheter according to claim 5, wherein the scoring member (2) is provided with a break point in a lengthwise direction, and a thickness of the scoring member (2) at the break point is less than a thickness at other parts thereof where no break point is arranged.

8. The scoring balloon catheter according to any one of claims 1-4, wherein the scoring member (2) is of a helical spring structure.

9. The scoring balloon catheter according to claim 1, wherein
when the torsion guide wire (6) is located in an interlayer, on an outer side wall, or on an inner side wall of the inner tube (3), the torsion guide wire (6) and the inner tube (3) are of an integrally formed structure or bonded together;
when the torsion guide wire (6) is located in an interlayer, on an outer side wall, or on an inner side wall of the outer tube (4), the torsion guide wire (6) and the outer tube (4) are of an integrally formed structure or bonded together; and
when the torsion guide wire (6) is located between the inner tube (3) and the outer tube

(4) two ends of the torsion guide wire (6) are connected to the balloon body (1) and the catheter seat (5) respectively, and part or a whole of the torsion guide wire (6) is connected to an outer side wall of the inner tube (3).

10. The scoring balloon catheter according to claim 1, wherein the balloon body (1) is of a structure with a cylindrical middle part and two tapered end parts, the cylindrical middle part is a working segment, and the two tapered end parts are non-working segments;
the scoring member (2) is arranged on the cylindrical middle part of the balloon body (1); and
when a plurality of scoring members (2) are provided, the plurality of scoring members (2) are evenly distributed in a circumferential direction on an outer surface of the balloon body (1).
